(19) United States
(12) Patent Application Publication
(10) Pub. No.: US 2019/0192176 A1
(43) Pub. Date: Jun. 27, 2019

(54) DILATION INSTRUMENT WITH GUIDE CATHETER TYPE SENSOR

(71) Applicant: Acclarent, Inc., Irvine, CA (US)

(72) Inventors: Jetmir Palushi, Irvine, CA (US); Itzhak Fang, Irvine, CA (US); Azhang Hamlekhan, Irvine, CA (US); David A. Smith, JR., Lake Forest, CA (US); Clayton Perry, Dallas, TX (US)

(21) Appl. No.: 15/852,470

(22) Filed: Dec. 22, 2017

(51) Int. Cl. A61B 17/24; A61M 25/00; A61M 29/02

(52) U.S. Cl. CPC A61B 17/24; A61M 25/0041; A61B 1/233; A61M 29/02; A61M 25/0097

(57) ABSTRACT

An apparatus includes a body assembly, a dilation catheter, a first guide catheter, and a guide catheter identification module. The body assembly includes a first electrical interface. The first guide catheter is configured to slidably receive the dilation catheter. The first guide catheter includes a first rigid shaft and a first guide catheter electrical contact. The first rigid shaft has a first rigid bend formed near the first distal end. The first rigid bend defines a first fixed bend angle. The first guide catheter is configured to be removably coupled with the body assembly such that the first guide catheter electrical contact engages the first electrical interface of the body assembly. The guide catheter identification module is operable to identify the first guide catheter based on engagement between the first guide catheter electrical contact and the first electrical interface of the body assembly.

### BACKGROUND

In some instances, it may be desirable to dilate an anatomical passageway in a patient. This may include dilation of ostia of paranasal sinuses (e.g., to treat sinusitis), dilation of the larynx, dilation of the Eustachian tube, dilation of other passageways within the ear, nose, or throat, etc. One method of dilating anatomical passageways includes using a guide wire and catheter to position an inflatable balloon within the anatomical passageway, then inflating the balloon with a fluid (e.g., saline) to dilate the anatomical passageway. For instance, the expandable balloon may be positioned within an ostium at a paranasal sinus and then be inflated, to thereby dilate the ostium by remodeling the bone adjacent to the ostium, without requiring incision of the mucosa or removal of any bone. The dilated ostium may then allow for improved drainage from and ventilation of the affected paranasal sinus. A system that may be used to perform such procedures may be provided in accordance with the teachings of U.S. Pub. No. 2011/0004057, entitled “Systems and Methods for Transnasal Dilation of Passageways in the Ear, Nose or Throat,” published Jan. 6, 2011, the disclosure of which is incorporated by reference herein. An example of such a system is the Relieva® Spin Balloon Sinuplasty™ System by Acclarent, Inc. of Irvine, Calif.

Image-guided surgery (IGS) is a technique where a computer is used to obtain a real-time correlation of the location of an instrument that has been inserted into a patient's body to a set of preoperatively obtained images (e.g., a CT or MRI scan, 3-D map, etc.), such that the computer system may superimpose the current location of the instrument on the preoperatively obtained images. In some IGS procedures, a digital tomographic scan (e.g., CT or MRI, 3-D map, etc.) of the operative field is obtained prior to surgery. A specially programmed computer is then used to convert the digital tomographic scan data into a digital map. During surgery, special instruments having sensors (e.g., electromagnetic coils that emit electromagnetic fields and/or are responsive to externally generated electromagnetic fields) mounted thereon are used to perform the procedure while the sensors send data to the computer indicating the current position of each surgical instrument. The computer correlates the data it receives from the instrument-mounted sensors with the digital map that was created from the preoperative tomographic scan. The tomographic scan images are displayed on a video monitor along with an indicator (e.g., crosshairs or an illuminated dot, etc.) showing the real-time position of each surgical instrument relative to the anatomical structures shown in the scan images. In this manner, the surgeon is able to know the precise position of each sensor-equipped instrument by viewing the video monitor even if the surgeon is unable to directly visualize the instrument itself at its current location within the body.

An example of an electromagnetic IGS systems that may be used in ENT and sinus surgery is the CARTO® 3 System by Biosense-Webster, Inc., of Irvine, Calif. When applied to functional endoscopic sinus surgery (FESS), balloon sinuplasty, and/or other ENT procedures, the use of IGS systems allows the surgeon to achieve more precise movement and positioning of the surgical instruments than can be achieved by viewing through an endoscope alone. As a result, IGS systems may be particularly useful during performance of FESS, balloon sinuplasty, and/or other ENT procedures where anatomical landmarks are not present or are difficult to visualize endoscopically.

While several systems and methods have been made and used in ENT procedures, it is believed that no one prior to the inventors has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1A depicts a perspective view of an exemplary dilation instrument assembly, with a guidewire in a proximal position, and with a dilation catheter in a proximal position;

FIG. 1B depicts a perspective view of the dilation instrument assembly of FIG. 1A, with the guidewire in a distal position, and with the dilation catheter in the proximal position;

FIG. 1C depicts a perspective view of the dilation instrument assembly of FIG. 1A, with the guidewire in a distal position, with the dilation catheter in a distal position, and with a dilator of the dilation catheter in a non-dilated state;

FIG. 1D depicts a perspective view of the dilation instrument assembly of FIG. 1A, with the guidewire in a distal position, with the dilation catheter in the distal position, and with a dilator of the dilation catheter in a dilated state;

FIG. 2 depicts a schematic view of an exemplary sinus surgery navigation system being used on a patient seated in an exemplary medical procedure chair;

FIG. 3 depicts a perspective view of portions of an exemplary dilation instrument in communication with the navigation system of FIG. 2;

FIG. 4 depicts a side elevational view of an exemplary guide catheter with a preformed bend angle suitable for insertion into a sphenoid sinus ostium of a patient's head;

FIG. 5 depicts a side elevational view of an exemplary alternative guide catheter with a preformed bend angle suitable for insertion into a Eustachian Tube of a patient's head;

FIG. 6 depicts a side elevational view of another exemplary alternative guide catheter with a preformed bend angle suitable for insertion into a frontal recess of a patient's head;

FIG. 7 depicts a side elevational view of still another exemplary alternative guide catheter with a preformed bend able suitable for insertion into a maxillary sinus ostium of a patient's head;

FIG. 8 depicts a perspective view of a distal portion of the handle assembly of the dilation instrument of FIG. 3 including a series of electrical contacts;

FIG. 9 depicts a flow diagram illustrating an exemplary algorithm that may be executed in association with the dilation instrument of FIG. 3 when one of the guide catheters of FIGS. 4-7 are assembled thereon;

FIG. 10 depicts a perspective view of an exemplary alternative dilation instrument assembly in communication with the navigation system of FIG. 2;

FIG. 11A depicts a cross-sectional side view of a distal portion of a bendable guide catheter of the dilation instrument FIG. 10, with the guide catheter in a straight configuration;

FIG. 11B depicts another cross-sectional side view of the distal portion of the bendable guide catheter of FIG. 11A, with the guide catheter in a bent configuration;

FIG. 12 depicts a flow diagram illustrating an exemplary algorithm that may be executed in association with the dilation instrument of FIG. 10 when the bendable guide catheter is actuated;

FIG. 13 depicts a side elevational view of a portion of another exemplary alternative dilation instrument assembly in communication with the navigation system of FIG. 2; and

FIG. 14 depicts a flow diagram illustrating an exemplary algorithm that may be executed in association with the dilation instrument of FIG. 13 when the bendable guide catheter is actuated.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

It will be appreciated that the terms “proximal” and “distal” are used herein with reference to a clinician gripping a handpiece assembly. Thus, an end effector is distal with respect to the more proximal handpiece assembly. It will be further appreciated that, for convenience and clarity, spatial terms such as “top” and “bottom” also are used herein with respect to the clinician gripping the handpiece assembly. However, surgical instruments are used in many orientations and positions, and these terms are not intended to be limiting and absolute.

It is further understood that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

I. Exemplary Dilation Catheter System

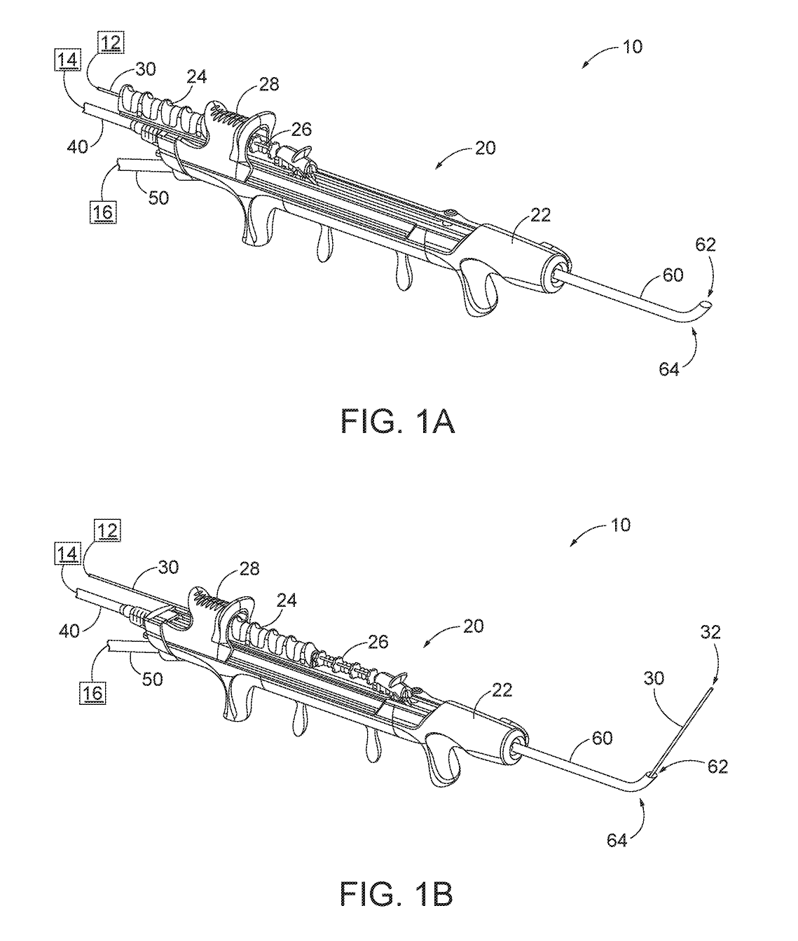

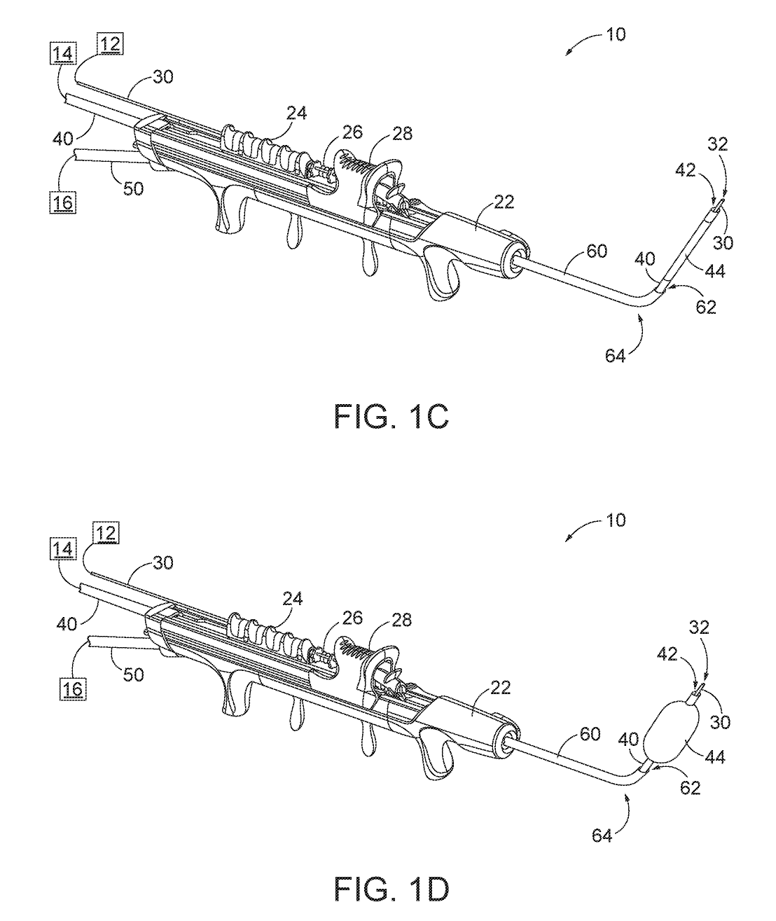

FIGS. 1A-1D show an exemplary dilation instrument assembly (10) that may be used to dilate the ostium of a paranasal sinus; to dilate some other passageway associated with drainage of a paranasal sinus; to dilate a Eustachian tube; or to dilate some other anatomical passageway (e.g., within the ear, nose, or throat, etc.). Dilation instrument assembly (10) of this example comprises a guidewire power source (12), an inflation source (14), an irrigation fluid source (16), and a dilation instrument (20). In some versions, guidewire power source (12) comprises a source of light. In some other versions, guidewire power source (12) is part of an IGS system as described below. Inflation source (14) may comprise a source of saline or any other suitable source of fluid. Irrigation fluid source (16) may comprise a source of saline or any other suitable source of fluid. Again, though, any other suitable source of fluid may be used. It should also be understood that irrigation fluid source (16) may be omitted in some versions.

Dilation instrument (20) of the present example comprise a handle body (22) with a guidewire slider (24), a guidewire spinner (26), and a dilation catheter slider (28). Handle body (22) is sized and configured to be gripped by a single hand of a human operator. Sliders (24, 28) and spinner (26) are also positioned and configured to be manipulated by the same hand that grasps handle body (22).

A guide catheter (60) extends distally from handle body (22). Guide catheter (60) includes an open distal end (62) and a bend (64) formed proximal to open distal end (62). Dilation instrument (20) is configured to removably receive several different kinds of guide catheters (60), each guide catheter (60) having a different angle formed by bend (64). Guide catheter (60) of the present example is formed of a rigid material (e.g., rigid metal and/or rigid plastic, etc.), such that guide catheter (60) maintains a consistent configuration of bend (64) during use of dilation instrument (20). In some versions, dilation instrument (20), is further configured to enable rotation of guide catheter (60), relative to handle body (22), about the longitudinal axis of the straight proximal portion of guide catheter (60), thereby further promoting access to various anatomical structures.

A guidewire (30) is coaxially disposed in guide catheter (60). Guidewire slider (24) is secured to guidewire (30). Translation of guidewire slider (24) relative to handle body (22) from a proximal position (FIG. 1A) to a distal position (FIG. 1B) causes corresponding translation of guidewire (30) relative to handle body (22) from a proximal position (FIG. 1A) to a distal position (FIG. 1B). When guidewire (30) is in a distal position, a distal portion of guidewire (30) protrudes distally from open distal end (62) of guide catheter (60). Guidewire spinner (26) is operable to rotate guidewire (30) about the longitudinal axis of guidewire (30). Guidewire spinner (26) is coupled with guidewire slider (24) such that guidewire spinner (26) translates longitudinally with guidewire slider (24). By way of example only, guidewire (30) may be configured in accordance with at least some of the teachings of U.S. Pat. No. 9,155,492, the disclosure of which is incorporated by reference herein. Other features and operabilities that may be incorporated into guidewire (30) will be apparent to those of ordinary skill in the art in view of the teachings herein.

A dilation catheter (40) is coaxially disposed in guide catheter (60). Dilation catheter slider (28) is secured to dilation catheter (40). Translation of dilation catheter slider (28) relative to handle body (22) from a proximal position (FIG. 1B) to a distal position (FIG. 1C) causes corresponding translation of dilation catheter (40) relative to handle body (22) from a proximal position (FIG. 1B) to a distal position (FIG. 1C). When dilation catheter (40) is in a distal position, a distal portion of dilation catheter (40) protrudes distally from open distal end (62) of guide catheter (60). Dilation catheter (40) of the present example comprises a non-extensible balloon (44) located just proximal to open distal end (42) of dilation catheter (40). Balloon (44) is in fluid communication with inflation source (14). Inflation source (14) is configured to communicate fluid (e.g., saline, etc.) to and from balloon (44) to thereby transition balloon (44) between a non-inflated state and an inflated state. FIG. 1C shows balloon (44) in a non-inflated state. FIG. 1D shows balloon (44) in an inflated state. In the non-inflated state, balloon (44) is configured to be inserted into a constricted anatomical passageway. In the inflated state, balloon (44) is configured to dilate the anatomical passageway in which balloon (44) is inserted. Other features and operabilities that may be incorporated into dilation catheter (40) will be apparent to those of ordinary skill in the art in view of the teachings herein.

II. Exemplary Image Guided Surgery Navigation System

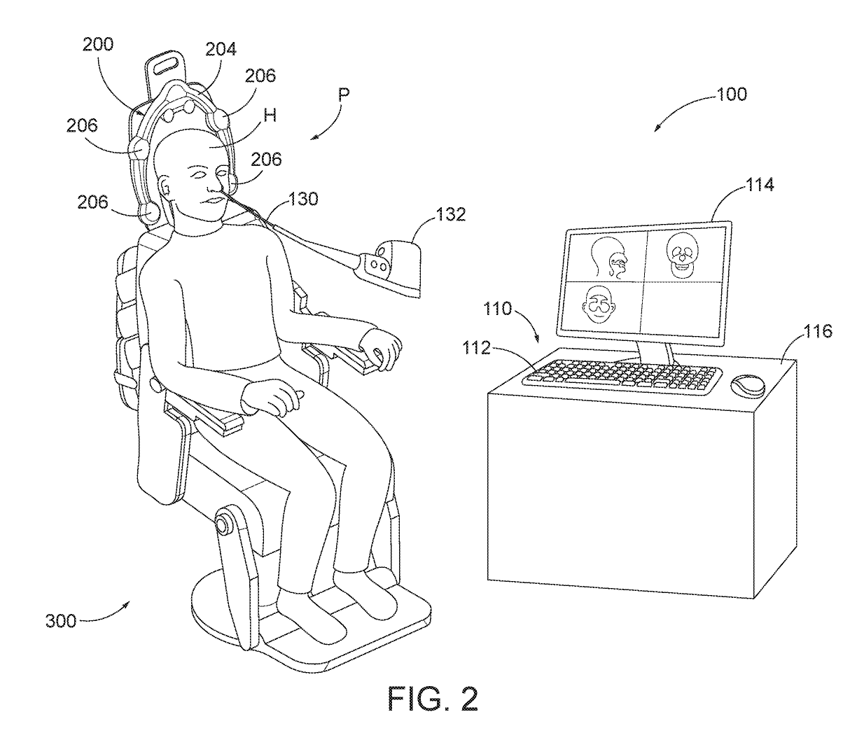

FIG. 2 shows an exemplary IGS navigation system (100) enabling an ENT procedure to be performed using image guidance. In some instances, IGS navigation system (100) is used during a procedure where dilation instrument assembly (10) is used to dilate the ostium of a paranasal sinus; or to dilate some other anatomical passageway (e.g., within the ear, nose, or throat, etc.). In addition to or in lieu of having the components and operability described herein IGS navigation system (100) may be constructed and operable in accordance with at least some of the teachings of U.S. Pat. No. 8,702,626, entitled “Guidewires for Performing Image Guided Procedures,” issued Apr. 22, 2014, the disclosure of which is incorporated by reference herein; U.S. Pat. No. 8,320,711, entitled “Anatomical Modeling from a 3-D Image and a Surface Mapping,” issued Nov. 27, 2012, the disclosure of which is incorporated by reference herein; U.S. Pat. No. 7,720,521, entitled “Methods and Devices for Performing Procedures within the Ear, Nose, Throat and Paranasal Sinuses,” issued May 18, 2010, the disclosure of which is incorporated by reference herein; U.S. Pat. Pub. No. 2014/0364725, entitled “Systems and Methods for Performing Image Guided Procedures within the Ear, Nose, Throat and Paranasal Sinuses,” published Dec. 11, 2014, the disclosure of which is incorporated by reference herein; U.S. Pub. No. 2016/0310042, entitled “System and Method to Map Structures of Nasal Cavity,” published Oct. 27, 2016; and U.S. Pat. Pub. No. 2011/0060214, entitled “Systems and Methods for Performing Image Guided Procedures within the Ear, Nose, Throat and Paranasal Sinuses,” published Mar. 10, 2011, the disclosure of which is incorporated by reference herein.

IGS navigation system (100) of the present example comprises a field generator assembly (200), which comprises set of magnetic field generators (206) that are integrated into a horseshoe-shaped frame (204). Field generators (206) are operable to generate alternating magnetic fields of different frequencies around the head of the patient. Field generators (206) thereby enable tracking of the position of a navigation guidewire (130) that is inserted into the head of the patient. Various suitable components that may be used to form and drive field generators (206) will be apparent to those of ordinary skill in the art in view of the teachings herein.

In the present example, frame (204) is mounted to a chair (300), with the patient (P) being seated in the chair (300) such that frame (204) is located adjacent to the head (H) of the patient (P). By way of example only, chair (300) and/or field generator assembly (200) may be configured and operable in accordance with at least some of the teachings of U.S. Patent App. No. 62/555,824, entitled “Apparatus to Secure Field Generating Device to Chair,” filed Sep. 8, 2017, the disclosure of which is incorporated by reference herein.

IGS navigation system (100) of the present example further comprises a processor (110), which controls field generators (206) and other elements of IGS navigation system (100). For instance, processor (110) is operable to drive field generators (206) to generate electromagnetic fields; and process signals from navigation guidewire (130) to determine the location of a sensor in navigation guidewire (130) within the head (H) of the patient (P). Processor (110) comprises a processing unit communicating with one or more memories. Processor (110) of the present example is mounted in a console (116), which comprises operating controls (112) that include a keypad and/or a pointing device such as a mouse or trackball. A physician uses operating controls (112) to interact with processor (110) while performing the surgical procedure.

A coupling unit (132) is secured to the proximal end of a navigation guidewire (130). Coupling unit (132) of this example is configured to provide wireless communication of data and other signals between console (116) and navigation guidewire (130). While coupling unit (132) of the present example couples with console (116) wirelessly, some other versions may provide wired coupling between coupling unit (132) and console (116). Various other suitable features and functionality that may be incorporated into coupling unit (132) will be apparent to those of ordinary skill in the art in view of the teachings herein.

Navigation guidewire (130) may be used as a substitute for guidewire (30) in dilation instrument (20) described above. Navigation guidewire (130) includes a sensor (not shown) that is responsive to movement within the fields generated by field generators (206). In the present example, the sensor of navigation guidewire (130) comprises at least one coil at the distal end of navigation guidewire (130). When such a coil is positioned within an electromagnetic field generated by field generators (206), movement of the coil within that magnetic field may generate electrical current in the coil, and this electrical current may be communicated along the electrical conduit(s) in navigation guidewire (130) and further to processor (110) via coupling unit (132). This phenomenon may enable IGS navigation system (100) to determine the location of the distal end of navigation guidewire (130) within a three-dimensional space (i.e., within the head (H) of the patient (P)). To accomplish this, processor (110) executes an algorithm to calculate location coordinates of the distal end of navigation guidewire (130) from the position related signals of the coil(s) in navigation guidewire (130).

Processor (110) uses software stored in a memory of processor (110) to calibrate and operate system (100). Such operation includes driving field generators (206), processing data from navigation guidewire (130), processing data from operating controls (112), and driving display screen (114). Processor (110) is further operable to provide video in real time via display screen (114), showing the position of the distal end of navigation guidewire (130) in relation to a video camera image of the patient's head (H), a CT scan image of the patient's head (H), and/or a computer generated three-dimensional model of the anatomy within and adjacent to the patient's nasal cavity. Display screen (114) may display such images simultaneously and/or superimposed on each other during the surgical procedure. Such displayed images may also include graphical representations of instruments that are inserted in the patient's head (H), such as navigation guidewire (130), such that the operator may view the virtual rendering of the instrument at its actual location in real time. By way of example only, display screen (114) may provide images in accordance with at least some of the teachings of U.S. Pub. No. 2016/0008083, entitled “Guidewire Navigation for Sinuplasty,” published Jan. 14, 2016, the disclosure of which is incorporated by reference herein. In the event that the operator is also using an endoscope, the endoscopic image may also be provided on display screen (114).

The images provided through display screen (114) may help guide the operator in maneuvering and otherwise manipulating instruments within the patient's head. When used as a substitute for guidewire (30) in dilation instrument assembly (10), navigation guidewire (130) may facilitate navigation of instrumentation of dilation instrument assembly (10) within the patient during performance of a procedure to dilate the ostium of a paranasal sinus; or to dilate some other anatomical passageway (e.g., within the ear, nose, or throat, etc.). It should also be understood that other components of dilation instrument assembly (10) may incorporate a sensor like the sensor of navigation guidewire (130), including but not limited to dilation catheter (40).

III. Exemplary Alternative Dilation Catheter System

As noted above, processor (110) of IGS navigation system (100) is operable to drive display screen (114) to render images of the head (H) of the patient (P). Such images may include various cross-sectional views of the head (H) of the patient (P), taken from a database or other collection of CT images and/or other kinds of images of the head (H) of the patient (P). The particular cross-sectional views (i.e., viewing angle, location of cross-sectional plane, etc.) that would be most beneficial to the operator may vary based on the targeted anatomical region. For instance, if the operator intends to dilate an ostium of a maxillary sinus, the operator may wish to observe cross-sectional views of the head (H) of the patient (P) with viewing angles and cross-sectional planes showing the maxillary sinus ostium and pathways leading thereto. If the operator intends to dilate frontal recess, the operator may wish to observe cross-sectional views of the head (H) of the patient (P) with viewing angles and cross-sectional planes showing the frontal and pathways leading thereto. In addition, settings such as the CT scan view layout displayed by console (116), the clipping angle, the available pressure profiles for a dilator of the dilation instrument, and/or the navigation instructions superimposed on display screen (114) may all vary depending on the designated anatomical passageway.

In some versions of IGS navigation system (100), the operator may need to manually manipulate operating controls (112) to obtain the view(s) and/or other settings that are best suited for guidance to the targeted anatomical region. This may require the operator to select from various options and make their selections by clicking on a mouse, typing on a keyboard, manually interacting with a touchscreen, and/or otherwise providing some form of manual input that is dedicated to selecting one or more views to be rendered on display screen (114) and/or other settings. This may add time to the procedure. It may therefore be desirable to automate the process of selecting view(s) and/or other settings that are best suited for guidance to the targeted anatomical region, prior to the commencement of an ENT procedure. The following describes several examples of how IGS navigation system (100) may automatically identify the targeted anatomical region, and thereby automatically select one or more view(s) to render on display screen (114) and/or other settings, based on the operator's assembly or manipulation of a dilation instrument, without requiring the operator to make any selections or otherwise provide input through operating controls (112).

It should be understood that the dilation instruments, guide catheters, and IGS systems described below may be readily incorporated into any of the various surgical procedures described in the various references described herein. Other suitable ways in which the below-described dilation instruments and guide catheters may be used will be apparent to those of ordinary skill in the art in view of the teachings herein.
A. Exemplary Array of Rigid Guide Catheters with Electrical Contacts

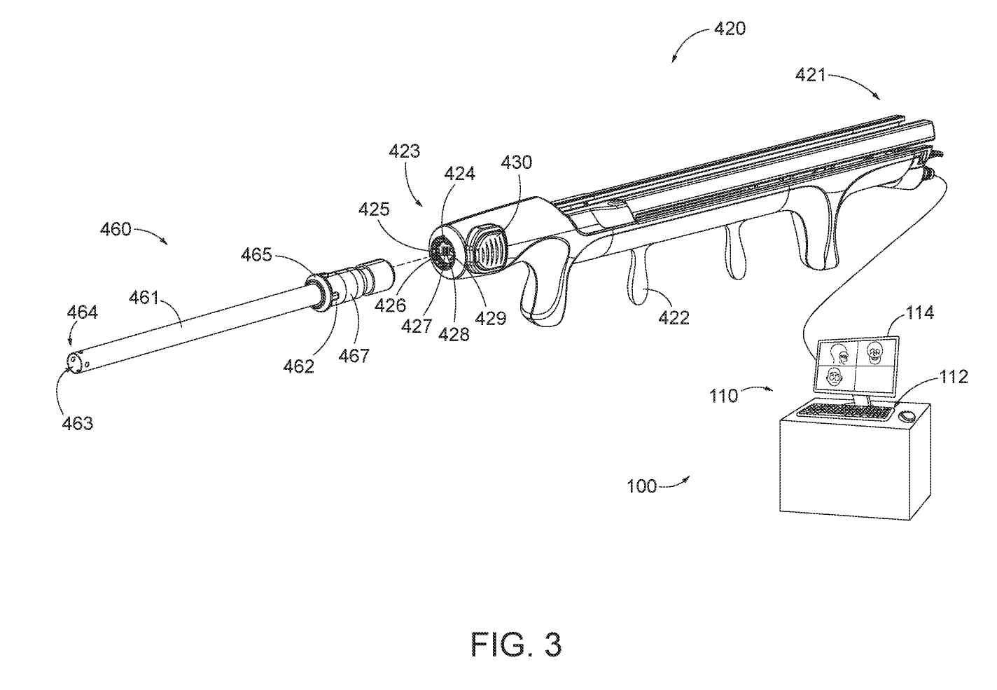

As noted above, a dilation instrument (20) may be configured to removably receive several different kinds of guide catheters (60) different angles formed by bend (64), with such different bend angles being selected to facilitate access to a particular anatomical region. FIG. 3 shows an exemplary dilation instrument assembly (400) that is operatively connected to IGS navigation system (100) through an electrical conduit (402). Although not shown, dilation instrument assembly (400) may be operatively connected to IGS navigation system (100) through other communicative methods, including but not limited to wireless transmission. Except as otherwise described below, dilation instrument assembly (400) is configured and operable just like dilation instrument assembly (10) described above. For instance, like dilation instrument assembly (10), dilation instrument assembly (400) comprises a dilation instrument (420) and a dilation catheter (460) that are configured and operable like dilation instrument (20) and dilation catheter (60) described above. Accordingly, dilation instrument assembly (400) of this example may be readily incorporated with IGS navigation system (100) described above.

Dilation instrument (420) includes a handle body (422) extending between a proximal portion (421) and a distal portion (423). While FIG. 3 does not show a guidewire slider, a guidewire spinner, or a dilation catheter slider, those of ordinary skill in the art will recognize that a guidewire slider, a guidewire spinner, and a dilation catheter slider like a guidewire slider (24), a guidewire spinner (26), and a dilation catheter slider (28), respectively, may be coupled with handle body (422). A guide catheter lock button (430) is oriented transversely near distal portion (423).

Distal portion (423) of dilation instrument (420) includes a guide port (424) and an annular engagement surface (425) extending along guide port (424). As will be described in greater detail below, guide port (424) is configured to receive a proximal hub (462, 472, 482, 492) of a guide catheter (460, 470, 480, 490) therein such that guide port (424) couples guide catheter (460, 470, 480, 490) to dilation instrument (420). As will also be described in greater detail below, annular engagement surface (425) is configured to engage an annular shoulder (465, 475, 485, 495) of proximal hub (462, 472, 482, 492) to thereby secure guide catheter (460, 470, 480, 490) to dilation instrument (420). A latching feature (not shown) retains guide catheter (460, 470, 480, 490) to handle body (422). Guide catheter lock button (430) is operable to disengage the latching feature, to thereby enable removal of guide catheter (460, 470, 480, 490) from handle body (422) to allow another guide catheter (460, 470, 480, 490) to be coupled with handle body (422).

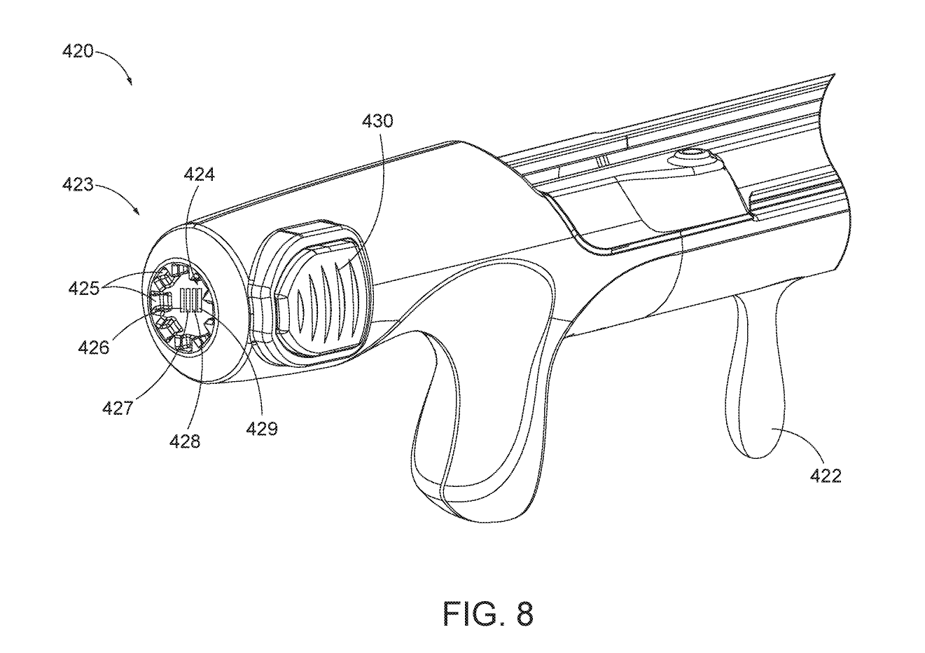

Dilation instrument (420) further includes one or more electrical contacts (426, 427, 428, 429) within guide port (424). As best seen in FIG. 8, dilation instrument (420) of the present example includes four electrical contacts (426, 427, 428, 429) positioned on each inner side of guide port (424). While not shown, identical electrical contacts (426, 427, 428, 429) may be located along an opposite inner wall of guide port (424) in longitudinal positions that correspond with the longitudinal positions of electrical contacts (426, 427, 428, 429) seen in FIG. 8, such that dilation instrument (420) comprises respective opposing pairs of electrical contacts (426, 427, 428, 429). While dilation instrument (420) of the present example comprises four pairs of electrical contacts (426, 427, 428, 429), some other versions may have more or fewer than four pairs of electrical contacts (426, 427, 428, 429). It should also be understood that electrical contacts (426, 427, 428, 429) may have other various shapes and sizes as will be apparent to one of ordinary skill in the art.

Each electrical contact (426, 427, 428, 429) is an electrical circuit component that is formed of an electrically conductive material. By way of example only, electrical contacts (426, 427, 428, 429) are formed of a metal, such as silver, gold alloys, or other suitable electrically conductive materials as will be apparent to those of ordinary skill in the art. As will be described in greater detail below, each electrical contact (426, 427, 428, 429) is located at a particular longitudinal position within guide port (424) that is configured to correspond with a particular contact ring (467, 477, 487, 497) positioned along guide catheter (460, 470, 480, 490) when guide catheter (460, 470, 480, 490) is coupled to dilation instrument (420). Electrical contacts (426, 427, 428, 429) are operable to communicate with contact ring (467, 477, 487, 497), respectively, once guide catheter (460, 470, 480, 490) is coupled to dilation instrument (420) to designate the type of guide catheter (460, 470, 480, 490) attached thereon.

As will be described in greater detail below, with contact ring (467, 477, 487, 497) communicating the type of guide catheter (460, 470, 480, 490) assembled to dilation instrument (420) through electrical contacts (426, 427, 428, 429), guide catheter (460, 470, 480, 490) effectively identifies the type of sinus or anatomical passageway that is to be treated. As a result, dilation instrument (420) is operable to communicate to processor (110) of IGS navigation system (100) the type of anatomical passageway that dilation instrument (420) is currently being targeted, such that processor (110) may automatically upload the most appropriate views and/or other settings that are particularly applicable for addressing the targeted anatomical passageway.

As merely an illustrative example, the preset settings that processor (110) may automatically upload onto the graphical user interface for use by an operator includes but is not limited to generating a one or more views (e.g., cross-sectional view(s)) and/or other graphical layout of the particular anatomical passageway to be treated on display screen (114); providing an automated inflation system that includes fluid pressure profiles for inflating expandable dilator (44) of dilation catheter (40) in accordance with the anatomical passageway to be dilated; providing one or more clipping angles; overlaying navigation instructions on display screen (114) to guide an operator in treating the designated anatomical passageway.

Those of ordinary skill in the art will understand that a “clipping angle” may include the angle of a clipping plane, where the three-dimensional image of the head (H) of the patient (P) is sliced in cross-section to show precisely where the tip of guidewire (130) (or some other sensor-equipped component of instrument (20)) is located in the head (H) of the patient (P). As instrument (20) moves through the head (H), the three-dimensional image may be continuously clipped or sliced by this clipping plane at the location of the tip of guidewire (130) (or some other sensor-equipped component of instrument (20)). By way of further example only, a clipping angle of 180° may provide a slice of the three-dimensional image of the head (H) yielding a coronal view on a persistent basis, such that the coronal view angle is maintained as the tip of guidewire (130) (or some other sensor-equipped component of instrument (20)) traverses through the three-dimensional space within the head (H). By way of further example only, a clipping angle of 90° may provide a slice of the three-dimensional image of the head (H) yielding a sagittal view.

Other kinds of preset settings that processor (110) may access and execute will be apparent to those of ordinary skill in the art in view of the teachings herein.

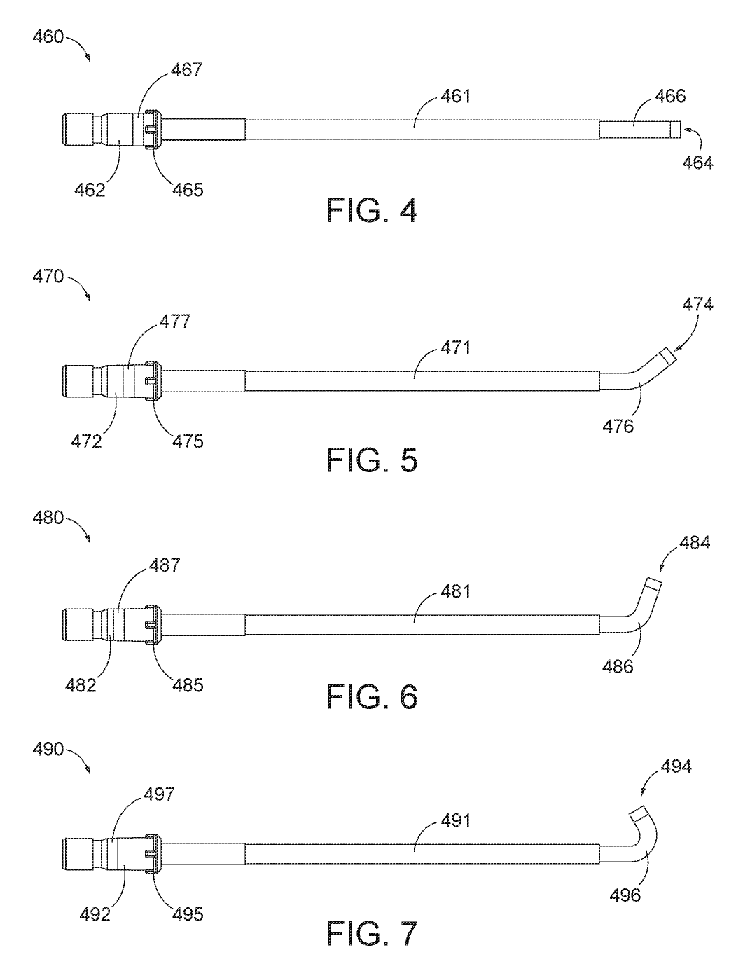

FIGS. 4-7 show various guide catheters (460, 470, 480, 490) that are configured to be removably coupled with dilation instrument (420). Each guide catheter (460, 470, 480, 490) is configured to guide a dilation catheter (e.g., dilation catheter (40)) for insertion into a particular anatomical passageway associated with the head (H) of a patient (P). Each guide catheter (460, 470, 480, 490) includes an elongate tubular shaft (461, 471, 481, 491) extending between a proximal hub (462, 472, 482, 492) and a distal portion (464, 472, 482, 492). By way of example only, and subject to the additional teachings below, guide catheters (460, 470, 480, 490) may be constructed and operable in accordance with at least some of the teachings of U.S. Pub. No. 2017/0056632, entitled “Dilation Catheter with Expandable Stop Element,” published Mar. 2, 2017, the disclosure of which is incorporated by reference herein.

Proximal hub (462, 472, 482, 492) is configured to be received and secured within guide port (424), such that proximal hub (462, 472, 482, 492) is operable to couple guide catheter (460, 470, 480, 490) to dilation instrument (420). Proximal hub (462, 472, 482, 492) is substantially rigid and ergonomically designed for insertion, location, and rotation into dilation instrument (420) with slight manipulations using a single hand. Proximal hub (462, 472, 482, 492) includes an annular shoulder (465, 475, 485, 495) at a distal end of proximal hub (462, 472, 482, 492). Annular shoulder (465, 475, 485, 495) is configured to abut against engagement surface (425) of guide port (424) to thereby prevent guide catheter (460, 470, 480, 490) from being further advanced into handle body (422) of dilation instrument (420).

Distal portion (464, 474, 484, 494) of the guide catheter (460, 470, 480, 490) may be constructed of a transparent material such as a polymer, including but not limited to nylon and PTFE, such that dilation catheter (40) is visible within distal portion (464, 474, 484, 494) and is more flexible than elongate tubular shaft (461, 471, 481, 491) of guide catheter (460, 470, 480, 490), respectively. Elongate tubular shaft (461, 471, 481, 491) defines a lumen (not shown) extending between proximal hub (462, 472, 482, 492) and distal portion (464, 474, 484, 494). Lumen (463, 473, 483, 493) is sized and shaped to slidably receive a guidewire and/or a balloon dilation catheter therein, such as guidewire (30) and dilation catheter (40), respectively, described above. Guide catheter (460, 470, 480, 490) may have any suitable length and/or diameter to receive guidewire (30) and dilation catheter (40) within lumen (463, 473, 483, 493) while remaining operable to fit within the particular anatomical passageway that guide catheter (460, 470, 480, 490) is configured to be inserted in, respectively.

In the present example, elongate tubular shaft (461, 471, 481, 491) is constructed of a stiff material such that guide catheter (460, 470, 480, 490) is configured to maintain a preformed bend (476, 486, 496) located along elongate tubular shaft (461, 471, 481, 491). By way of example only, elongate tubular shaft (461, 471, 481, 491) of guide catheter (460, 470, 480, 490) may be formed of stainless steel, rigid plastic, and/or any other suitable materials as will be apparent to those of ordinary skill in the art.

Each guide catheter (460, 470, 480, 490) further includes a contact ring (467, 477, 487, 497) positioned at a particular longitudinal position on proximal hub (462, 472, 482, 492). The locations of contact rings (467, 477, 487, 497) differ from each other, such that the respective position of contact rings (467, 477, 487, 497) are configured to correspond to a particular longitudinal position of a corresponding pair of electrical contacts (426, 427, 428, 429) in guide port (424). In other words, contact ring (467, 477, 487, 497) is located at a particular longitudinal position on proximal hub (462, 472, 482, 492) that aligns with a corresponding pair of electrical contacts (426, 427, 428, 429) in guide port (424) of handle body (422) when guide catheter (460, 470, 480, 490) is coupled to dilation instrument (420). Contact ring (467, 477, 487, 497) is an electrical circuit component that is formed of an electrically conductive material. By way of example only, contact ring (467, 477, 487, 497) may be formed of a metal, such as silver, gold alloys, or other suitable electrically conductive materials as will be apparent to those of ordinary skill in the art.

As seen in FIGS. 4-7, contact rings (467, 477, 487, 497) extend around a lateral perimeter of proximal hubs (462, 472, 482, 492) such that contact rings (467, 477, 487, 497) are disposed about the outer diameter of proximal hubs (462, 472, 482, 492). Contact ring (467, 477, 487, 497) is fixedly secured to proximal hub (462, 472, 482, 492) such that guide catheter (460, 470, 480, 490) is configured to only align with the particular pair of electrical contacts (426, 427, 428, 429) that corresponds with the position of electrical ring (467, 477, 487, 497) when proximal hub (462, 472, 482, 492) is inserted into guide port (424).

As shown in FIG. 4, elongate tubular shaft (461) of guide catheter (460) may have a preformed bend (466) along distal portion (464) that is configured to facilitate access into a first particular anatomical passageway. By way of example only, preformed bend (466) has an angle of approximately 0 degrees and is configured to facilitate access into a patient's sphenoid sinus ostium. Other suitable angles of bend (466) may be formed along distal portion (464) as will be apparent to those of ordinary skill in the art. Contact ring (467) is located at a first longitudinal position along proximal hub (462), immediately adjacent to annular shoulder (465) such that contact ring (467) abuts against annular shoulder (465). Contact ring (467) is configured to align with electrical contact (426) of dilation instrument (420) such that contact ring (467) encounters and/or comes into direct contact with electrical contacts (426) when proximal hub (462) is fully inserted into guide port (424) and guide catheter (460) is effectively coupled to dilation instrument (420). Although contact ring (467) may temporarily encounter other electrical contacts (427, 428, 429) as proximal hub (462) is initially inserted into guide port (424), contact ring (467) is configured to contact only electrical contacts (426) once proximal hub (462) is fully seated in guide port (424) such that annular shoulder (465) abuts against engagement surface (425).

As shown in FIG. 5, elongate tubular shaft (471) of guide catheter (470) may have a preformed bend (476) along distal portion (474) that is configured to facilitate access into a second particular anatomical passageway. By way of example only, preformed bend (476) has an angle of approximately 30 degrees to facilitate access into a patient's Eustachian Tube. Other suitable angles of bend (476) may be formed along distal portion (474) as will be apparent to those of ordinary skill in the art. Contact ring (477) is located at a second longitudinal position along proximal hub (462) that is offset from the first position described above, such that contact ring (477) is positioned along proximal hub (472) of guide catheter (470) proximally relative to the position of contact ring (467) on proximal hub (462) of guide catheter (460). Contact ring (477) is configured to align with electrical contacts (427) of dilation instrument (420) such that contact ring (477) contacts electrical contacts (427) when proximal hub (472) is fully seated in guide port (424). Although contact ring (477) may temporarily encounter other electrical contacts (426) as proximal hub (472) is initially inserted into guide port (424), contact ring (477) is configured to contact only electrical contacts (427) once proximal hub (472) is fully seated in guide port (424) such that annular shoulder (475) abuts against engagement surface (425).

As shown in FIG. 6, elongate tubular shaft (481) of guide catheter (480) may have a preformed bend (486) along distal portion (484) that is configured to facilitate access into a third particular anatomical passageway. By way of example only, preformed bend (486) has an angle of approximately 70 degrees to facilitate access into a patient's frontal recess. Other suitable angles for bend (486) may be formed along distal portion (484) as will be apparent to those of ordinary skill in the art. Contact ring (487) is located at a third longitudinal position along proximal hub (482) that is offset from the first and second positions described above, such that contact ring (487) is positioned along proximal hub (482) of guide catheter (480) proximally relative to the position of contact rings (467, 477) on proximal hubs (462, 472), respectively. Contact ring (487) is configured to align with electrical contacts (428) of dilation instrument (420) such that contact ring (487) contacts electrical contact (428) when proximal hub (482) is fully seated in guide port (424). Although contact ring (487) may temporarily encounter other electrical contacts (426, 427) as proximal hub (482) is initially inserted into guide port (424), contact ring (487) is configured to contact only electrical contacts (428) once proximal hub (482) is fully seated in guide port (424) such that annular shoulder (485) abuts against engagement surface (425).

As shown in FIG. 7, elongate tubular shaft (491) of guide catheter (490) may have a preformed bend (496) along distal portion (494) that is configured to facilitate access into a fourth particular anatomical passageway. By way of example only, preformed bend (496) has an angle of approximately 90 degrees to 110 degrees to facilitate access into a patient's maxillary sinus ostium. Other suitable angles for bend (496) may be formed along distal portion (494) as will be apparent to those of ordinary skill in the art. Contact ring (497) is located at a fourth longitudinal position along proximal hub (492) that is offset from the first, second, and third positions described above, such that contact ring (497) is positioned along proximal hub (492) of guide catheter (490) proximally relative to the position of contact rings (467, 477, 487) on proximal hubs (462, 472, 482). Contact ring (497) is configured to align with electrical contacts (429) of dilation instrument (420) such that contact ring (497) contacts electrical contacts (429) when proximal hub (492) is fully seated in guide port (424). Although contact ring (497) may temporarily encounter other electrical contacts (426, 427, 428) as proximal hub (492) is initially inserted into guide port (424), contact ring (497) is configured to contact only electrical contacts (429) once proximal hub (492) is fully seated in guide port (424) such that annular shoulder (495) abuts against engagement surface (425).

Each contact ring (467, 477, 487, 497) is configured to complete an electrical circuit between the pair of electrical contact (426, 427, 428, 429) that are located at the longitudinal position corresponding to contact ring (467, 477, 487, 497) when guide catheter (460, 470, 480, 490) is coupled with dilation instrument (420). In other words, when guide catheter (460) is coupled with dilation instrument (420), contact ring (467) completes a circuit between electrical contacts (426); when guide catheter (470) is coupled with dilation instrument (420), contact ring (477) completes a circuit between electrical contacts (427); when guide catheter (480) is coupled with dilation instrument (420), contact ring (487) completes a circuit between electrical contacts (428); and when guide catheter (490) is coupled with dilation instrument (420), contact ring (497) completes a circuit between electrical contacts (429). In some versions, dilation instrument (420) is configured to automatically apply a voltage to all electrical contacts (426, 427, 428, 429) when a guide catheter (460, 470, 480, 490) is fully seated in guide port (424), to determine which electrical contacts (426, 427, 428, 429) are being electrically coupled by a corresponding contact ring (467, 477, 487, 497). This automatic application of a voltage may be accomplished using a switch that is activated in response to full insertion of a proximal hub (462, 472, 482, 492) in guide port (424). Various suitable ways in which this may be carried out will be apparent to those of ordinary skill in the art in view of the teachings herein.

When a circuit is completed between a particular pair of electrical contacts (426, 427, 428, 429) via a corresponding contact ring (467, 477, 487, 497), an electrical current is allowed to flow between the electrical contacts (426, 427, 428, 429) of that particular pair via the corresponding contact ring (467, 477, 487, 497). A processor (e.g., processor (110)) or other circuit component that is located in dilation instrument (420) or in IGS system (100) is operable to detect which pair of electrical contacts (426, 427, 428, 429) has the completed circuit, and thereby identifies which particular guide catheter (460, 470, 480, 490) is coupled with dilation instrument (420). IGS system (100) is thereby operable to determine which particular anatomical passageway is being targeted. Processor (110) may then select the most appropriate view(s) and/or other settings that are best suited for that particular anatomical passageway.
B. Exemplary Operation of Dilation Instrument with Rigid Guide Catheters with Electrical Contacts

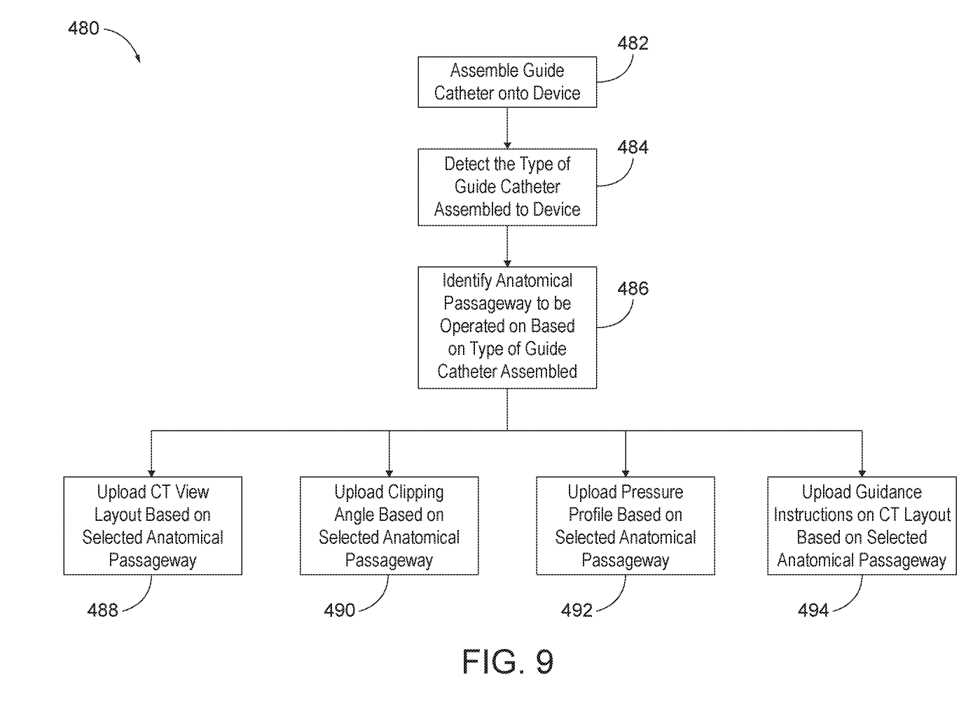

FIG. 9 shows a flow diagram illustrating steps of an exemplary method (480) that may be executed using dilation instrument assembly (400) and IGS navigation system (100). At step (482), an operator of dilation instrument assembly (400) initially identifies the particular anatomical passageway of a patient that is to be treated to thereby determine which guide catheter (460, 470, 480, 490) is most appropriate to use for that procedure. For instance, an operator that seeks to treat a sphenoid sinus ostium may select guide catheter (460) to assemble onto dilation instrument (420), as guide catheter (460) is configured to facilitate access to a patient's sphenoid sinus ostium. Alternatively, an operator that seeks to treat an Eustachian Tube may select guide catheter (470) to assemble onto dilation instrument (420), as guide catheter (470) is configured to facilitate access to a patient's Eustachian Tube.

Upon selecting the appropriate guide catheter (460, 470, 480, 490) based on the respective bend angles (466, 476, 486, 496) of each guide catheter (460, 470, 480, 490) in light of the particular anatomical passageway that is to be treated with dilation instrument assembly (400), the operator assembles dilation instrument assembly (400) by directing proximal hub (462, 472, 482, 492) of guide catheter (460, 470, 480, 490) proximally toward handle body (422) of dilation instrument (420). In this instance, proximal hub (462, 472, 482, 492) is inserted into guide port (424) until annular shoulder (465, 475, 485, 495) abuts against engagement surface (425) to thereby securely fasten guide catheter (460, 470, 480, 490) to dilation instrument (420). With proximal hub (462, 472, 482, 492) fully inserted into guide port (424), the respective contact ring (467, 477, 487, 497) of the particular guide catheter (460, 470, 480, 490) that is assembled to dilation instrument (420) will encounter the pair of electrical contacts (426, 427, 428, 429) in guide port (424) that is configured to align with that particular guide catheter (460, 470, 480, 490). In other words, depending on which guide catheter (460, 470, 480, 490) the operator determined to assemble to dilation instrument (420), contact ring (467, 477, 487, 497) will contact the corresponding electrical contacts (426, 427, 428, 429), thereby completing a circuit between the pair of electrical contacts (426, 427, 428, 429).

At step (484), dilation instrument assembly (400) and/or IGS system (100) detects the type of guide catheter (460, 470, 480, 490) assembled onto dilation instrument (420) as indicated to dilation instrument assembly (400) through the connection of contact ring (467, 477, 487, 497) and the corresponding electrical contacts (426, 427, 428, 429). As a result, dilation instrument assembly (400) and/or IGS system (100) identifies the particular anatomical passageway that is to be treated by dilation instrument (420) based on the type of guide catheter (460, 470, 480, 490) currently attached to dilation instrument (420), as seen at step (486).

Upon receiving the data indicating which particular anatomical passageway is being targeted, processor (110) processes the information and communicates with one or more memories to upload the respective preset settings that are associated with the anatomical passageway that is to be treated. Processor (110) uses software stored in a memory to upload, calibrate, and/or operate IGS navigation system (100) with the selected preset settings. Steps (488, 489, 492, 494) show various optional responses that processor (110) may provide in response to determining which particular anatomical passageway is being targeted. These steps (488, 489, 492, 494) may be performed simultaneously or in a sequence. As another variation, one or more of these steps (488, 489, 492, 494) may be omitted. Still other kinds of automated responses that may be provided in response to detection of the targeted anatomical passageway will be apparent to those of ordinary skill in the art in view of the teachings herein.

As noted above, IGS navigation system (100) tracks the position of the tip of guidewire (130) (or some other sensor-equipped component of instrument (420)) and projects that position virtually onto a preoperative CT scan image of the head (H) of the patient (P), rendered in triple orthogonal projections and/or three-dimensional views, updating the position projection in real time as the sensor-equipped component moves within the head (H) of the patient (P). In view of this, those of ordinary skill in the art will recognize that treatment of different anatomical regions in the head (H) may warrant use of different view layouts of the CT scan view, based on the targeted anatomy, to most effectively show the relevant anatomy to the operator. Thus, at step (488), IGS navigation system (100) automatically uploads the particular CT scan view layout that corresponds to the particular anatomical passageway to be treated onto display screen (114). This may include one or more views showing particular cross-sectional planes and viewing angles of the targeted anatomical passageway and/or one or more pathways leading to the targeted anatomical passageway.

At step (490), IGS navigation system (100) uploads the particular clipping angle that corresponds to the particular anatomical passageway to be treated.

In some versions, dilation instrument (420) is coupled with an automated inflator system that is operable to communicate fluid to and from dilation catheter (40) in an automated fashion. Such an automated inflator system may provide different fluid pressure profiles based on the targeted anatomical passageway (e.g., a different inflation pressure for dilating a sinus ostium versus dilating a Eustachian tube). By way of example only, such an automated inflator system may be constructed and operable in accordance with at least some of the teachings of U.S. Pub. No. 2016/0058985, entitled “Automated Inflator for Balloon Dilator,” published Mar. 3, 2016, the disclosure of which is incorporated by reference herein. Step (492) is associated with versions where an automated inflator system is used. In particular, at step (492), IGS navigation system (100) uploads the particular fluid pressure profiles for use by balloon (44) of dilation catheter (40) that correspond to the particular anatomical passageway to be treated.

In some versions, IGS navigation system (100) is operable to provide active navigation instructions to the operator via display screen (114), based on the particular anatomical passageway that is being targeted. By way of example only, such active navigation instructions may be provided in accordance with at least some of the teachings of U.S. Pub. No. 2016/0008083, entitled “Guidewire Navigation for Sinuplasty,” published Jan. 14, 2016, the disclosure of which is incorporated by reference herein. Step (494) is associated with versions where active navigation instructions are provided. In particular, at step (494), IGS navigation system (100) uploads the particular guidance instructions overlaid on the CT scan view layout displayed on screen (114) based on the particular anatomical passageway to be treated.
C. Exemplary Deflectable Guide with Linear Position Sensor

In some instances, it may be desirable to use a dilation instrument with a single, adjustable guide catheter that may be used to dilate various different anatomical passageways (e.g., frontal sinus ostium, frontal recess, maxillary sinus ostium, sphenoid sinus ostium, ethmoid sinus ostium, Eustachian tube, etc.), rather than requiring separate guide catheters, such as guide catheters (460, 470, 480, 490) described above, to be assembled onto the dilation instrument for treating different anatomical passageways. Providing a dilation instrument that has an adjustable guide catheter may be beneficial to minimize the total number of device components required to perform an ENT procedure on a patient.

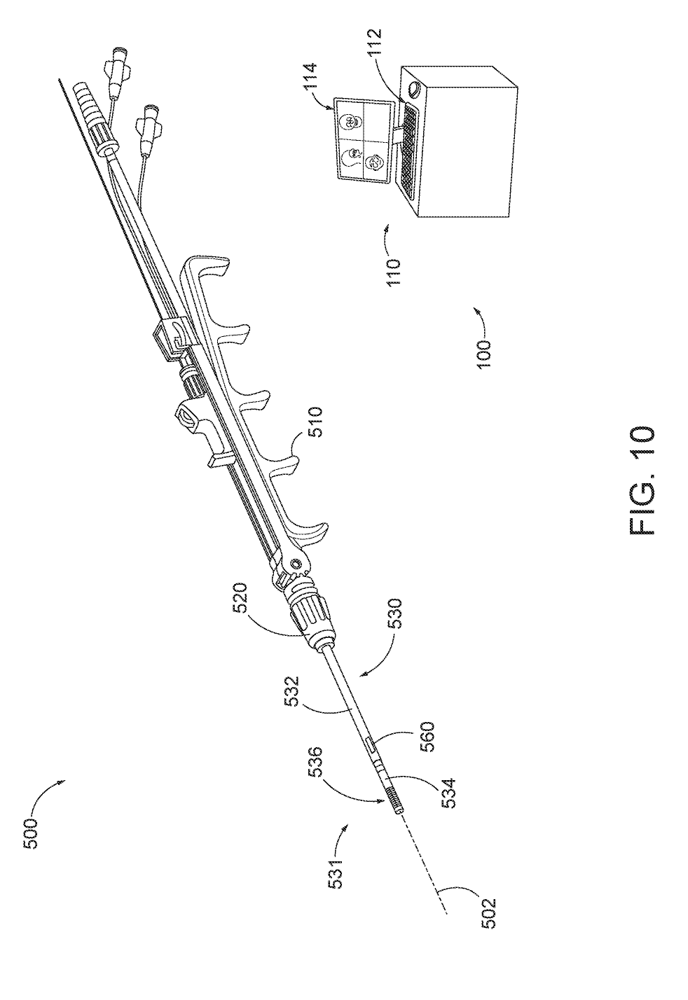

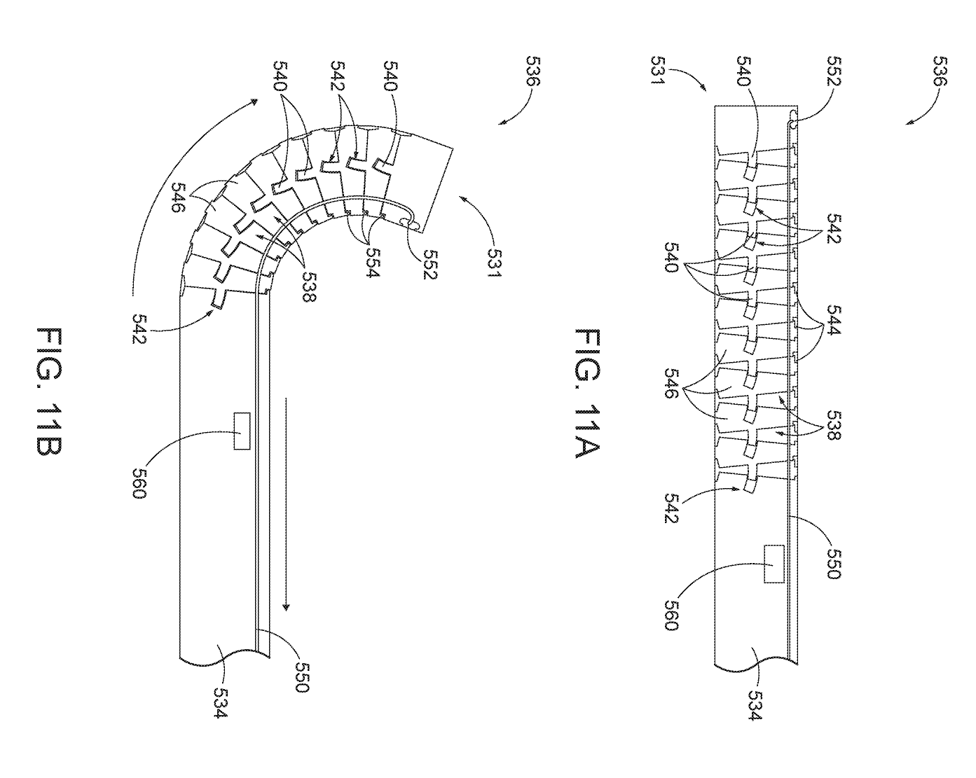

FIG. 10 shows an exemplary dilation instrument (500) that is configured to enable adjustment of an integral guide catheter for use with various different anatomical passageways. Dilation instrument (500) is configured to be operatively connected to IGS navigation system (100) via wire or wirelessly. As will be described in greater detail below, dilation instrument (500) is configured and operable substantially similar to dilation instrument assembly (400), such that dilation instrument (500) is operable to provide data indicating the type of anatomical passageway that is to be treated by dilation instrument (500) to processor (110) of IGS navigation system (100).

Dilation instrument (500) comprises a handle assembly (510), a deflection control knob (520), and a shaft assembly (530). Shaft assembly (530) extends distally from handle assembly (510) along a longitudinal axis (502). Unlike dilation instrument assembly (400) described above, which includes guide catheters (460, 470, 480, 490) that have a rigid configuration with a fixed bend angle (466, 476, 486, 496), dilation instrument (500) comprises a shaft assembly (530) that is adjustable to form varying lateral bend angles. By way of example only, dilation instrument (500) may be constructed and operable in accordance with at least some of the teachings of U.S. Patent App. No. 62/555,841, entitled “Adjustable Instrument for Dilation of Anatomical Passageway,” filed on Sep. 8, 2017, the disclosure of which is incorporated by reference herein.

Deflection control knob (520) is positioned at a distal end of handle assembly (510) and a proximal end of shaft assembly (530). Deflection control knob (520) is configured to deflect and/or laterally bend shaft assembly (530) in response to rotation of control knob (520) relative to handle assembly (510). Shaft assembly (530) includes a rigid shaft member (532) and a flexible shaft member (534) that extend distally to a distal end (531). Flexible shaft member (534) is secured to rigid shaft member (532), is coaxially aligned with rigid shaft member (532), and is positioned distally in relation to rigid shaft member (532). As will be described in greater detail below, shaft assembly (300) may include an integral linear position sensor (560) to detect the longitudinal position of a push-pull wire (550) to determine the angle of deflection at flexible shaft member (534), and thereby indicate the particular type of anatomical passageway that dilation instrument (500) is being used to treat.

As best seen in FIGS. 11A-11B, flexible shaft member (532) includes a flex section (536) that is formed by a series of ribs (546), which are separated by a series of notches (538). Notches (538) are generally V-shaped, with a circular opening at the vertex of each “V.” Notches (538) also include tab portions (540) that fit in corresponding sub-notches (542). The top of each “V” includes a set of stop features (544). As shown in FIG. 11A, when flex section (534) is in a straight configuration, tab portions (540) are disposed in corresponding sub-notches (542) but are not fully seated in sub-notches (542). When flex section (536) is in a straight configuration, stop features (544) are separated from each other.

FIG. 11B shows flex section (536) in a fully bent configuration. In this state, tab portions (540) are fully seated in sub-notches (542) and stop features (544) are engaged with each other. During the transition between the states shown in FIGS. 11A-11B, tab portions (540) and sub-notches (542) may cooperate to ensure that flex section (546) bends in a consistent fashion, with sufficient lateral stability; and that flex section (546) provides a consistent and stable bent or straight state. Those of ordinary skill in the art will appreciate that flex section (536) may achieve various other bend angles between the straight configuration shown in FIG. 11A and the bend angle shown in FIG. 11B.

Various suitable forms that flex section (536) may take will be apparent to those of ordinary skill in the art in view of the teachings herein. By way of example only, flex section (536) may be constructed and operable in accordance with at least some of the teachings of U.S. Pat. App. No. 62/490,235, entitled “Deflectable Guide for Medical Instrument,” filed Apr. 26, 2017, the disclosure of which is incorporated by reference herein.

Dilation instrument (500) further includes a push-pull wire (550) disposed within shaft members (532, 534). Push-pull wire (550) is configured to provide controlled bending of flex section (536). As shown in FIGS. 11A-11B, a distal end (552) of push-pull wire (550) is secured to distal end (531) of flexible shaft member (534), distal to flex section (536). Push-pull wire (550) is disposed near the tops of the “V”s of notches (538). Thus, when push-pull wire (550) is pulled proximally, flex section (536) will bend to a deflected configuration. When push-pull wire (550) is pushed distally, flex section (536) will bend toward a straight configuration.

A proximal end of push-pull wire (550) is coupled with deflection control knob (520) via an inner cam barrel (not shown). The proximal end of push-pull wire (550) is disposed in a bore of the cam barrel and passes transversely through the cam barrel, with the cam barrel being positioned and within control knob (520). The inner cam barrel is coupled with rigid shaft member (532) such that the cam barrel is allowed to slide longitudinally along rigid shaft member (532); yet the cam barrel is prevented from rotating about rigid shaft member (532). The cam barrel includes a pair of cam slots (not shown) that extend along generally helical paths to provide a transition path for inner pins (not shown) of deflection control knob (520) to travel through. Thus, the cam barrel will translate longitudinally in response to rotation of deflection control knob (520). This translation of the cam barrel by control knob (520) causes the simultaneous translation of push-pull wire (550), which will thereby cause straightening or bending of flex section (536). Such operability may be provided in accordance with at least some of the teachings of U.S. Patent App. No. 62/555,841, entitled “Adjustable Instrument for Dilation of Anatomical Passageway,” filed on Sep. 8, 2017, the disclosure of which is incorporated by reference herein.

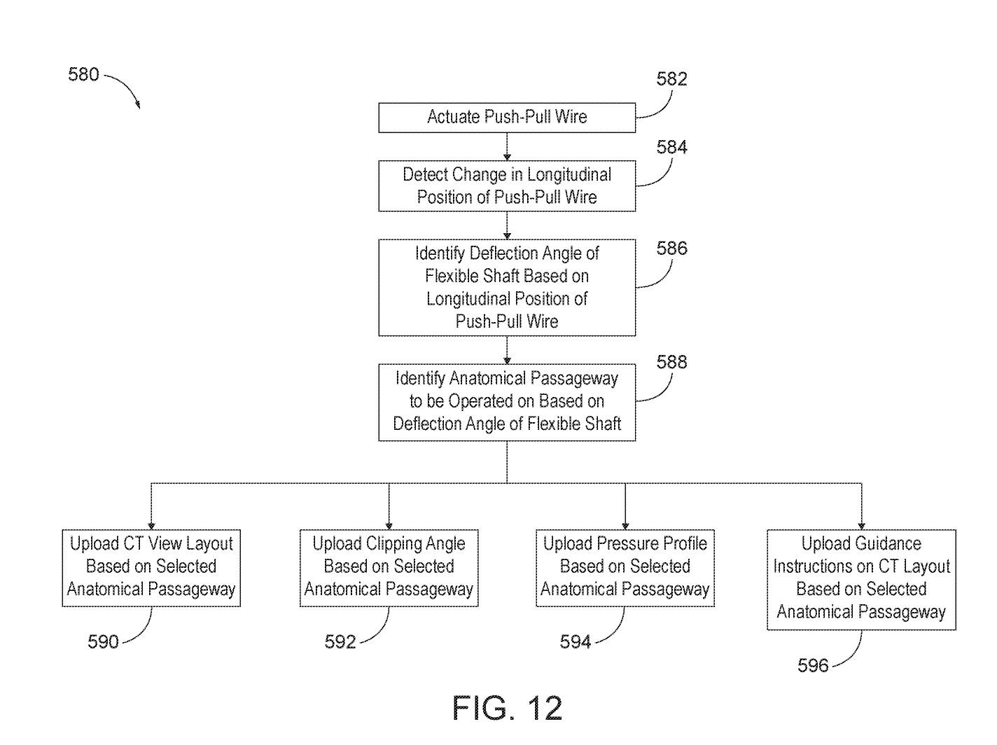

As best seen in FIGS. 11-12, shaft assembly (300) further includes a linear position sensor (560) that is integrally positioned along flexible shaft member (534) proximally relative to flex section (536). In this instance, linear position sensor (560) is fixedly secured to flexible shaft member (534) at a longitudinally fixed position such that linear position sensor (560) does not translate or deflect in response to deflection of flex section (536) of flexible shaft member (534).

Linear position sensor (560) is configured to monitor and/or sense a longitudinal position of push-pull wire (550) relative to sensor (560). The longitudinal position of push-pull wire (550) relative to sensor (560) will be indicative of the bend angle of flex section (536), which will in turn be indicative of the anatomical passageway that is being targeted by dilation instrument (500). A processor in dilation instrument (500) and/or in IGS navigation system (100) (e.g., processor (110)) may process signals from linear position sensor (560) to determine the bend angle of flex section (536), and thereby determine the anatomical passageway that is being targeted by dilation instrument (500). Processor (110) is operable to upload then preset settings that correspond to treating that particular anatomical passageway.

While linear position sensor (560) is shown as being positioned at a particular location in FIGS. 11A-11B, linear position sensor (560) may instead be fixedly secured anywhere along the length of shaft assembly (530); or anywhere else that linear position sensor (560) is operable to track longitudinal movement of push-pull wire (550). Various suitable forms that linear position sensor (560) may take will be apparent to those of ordinary skill in the art in view of the teachings herein.
D. Exemplary Operation of Dilation Instrument with Deflectable Guide with Linear Position Sensor

FIG. 12 shows a flow diagram illustrating steps of an exemplary method (580) that may be executed using dilation instrument (500) and IGS navigation system (100). At step (582), an operator of dilation instrument (500) determines the extent to which it is desired to deflect flex section (536) of flexible shaft member (534). The operator rotates control knob (520) to provide deflection at flex section (536) relative to handle assembly (510), to thereby achieve a desired bend angle.

If control knob (520) is not rotated, flex section (536) maintains a straight configuration as shown in FIG. 10 such that shaft assembly (530) is shaped to easily guide a guidewire (not shown) and a dilation catheter (not shown) into a first particular anatomical passageway. By way of example only, a flex section (536) having a bend angle of approximately 0 degrees may be particularly suitable for accessing and treating a sphenoid sinus ostium. Alternatively, if the operator rotates control knob (520), push-pull wire (550) is translated proximally toward handle assembly (510). In this instance, as seen in FIG. 11B, push-pull wire (550) deflects distal end (552) laterally thereby causing partial deflection of flex section (536) such that flex section (536) is no longer in a straight configuration. Push-pull wire (550) is pulled proximally until deflection control knob (520) is no longer rotated, thereby forming a particular bend angle along flexible shaft member (534).

As merely illustrative examples, an operator that seeks to treat an Eustachian Tube may rotate control knob (520) to an extent until push-pull wire (550) pulls distal end (552) toward proximal end (554) to thereby form a bend at flex section (536) that is configured to facilitate access to a patient's Eustachian Tube. By way of example only, push-pull wire (550) may be pulled proximally to a linear extent that corresponds with flex section (536) having a bend angle that is approximately 30 degrees. Alternatively, an operator that seeks to treat a frontal recess may rotate control knob (520) to an extent until push-pull wire (550) pulls distal end (552) toward proximal end (554) to thereby form a bend at flex section (536) that is configured to facilitate to a patient's frontal recess, such as approximately 70 degrees. Similarly, an operator that seeks to treat a patient's maxillary sinus ostium may rotate control knob (520) to an extent until push-pull wire (550) pulls distal end (552) toward proximal end (554) to thereby form a bend at flex section (536) that is configured to facilitate access to a patient's maxillary sinus ostium, such as approximately 90 degrees to 110 degrees.

As the operator rotates control knob (520) to achieve a desired bend angle, at step (584), linear position sensor (560) detects the longitudinal change in position of push-pull wire (550) relative to the fixed position of sensor (560) along shaft assembly (530). With sensor (560) having detected the extent of longitudinal translation of push-pull wire (550) toward handle assembly (510), a processor in dilation instrument (500) and/or in IGS navigation system (100) (e.g., processor (110)) determines the deflection angle formed at flex section (536) as seen at step (586). In particular, the further push-pull wire (550) is pulled proximally toward handle assembly (510), the greater change in longitudinal position linear position sensor (560) will detect. Accordingly, linear position sensor (560) will recognize that the extent of longitudinal translation of push-pull wire (550) in the proximal direction is directly proportional to a greater bend formed at flex section (536). In this instance, as seen at step (588), a processor in dilation instrument (500) and/or in IGS navigation system (100) (e.g., processor (110)) identifies the particular type of anatomical passageway that dilation instrument (500) is being used to treat, based on the deflection angle that was identified based on data from sensor (560).

Upon receiving the data indicating which particular anatomical passageway is being targeted, processor (110) processes the information and communicates with one or more memories to upload the respective preset settings that are associated with the anatomical passageway that is to be treated. Processor (110) uses software stored in a memory to upload, calibrate, and/or operate IGS navigation system (100) with the selected preset settings. Steps (590, 592, 594, 596) show various optional responses that processor (110) may provide in response to determining which particular anatomical passageway is being targeted. These steps (590, 592, 594, 596) may be performed simultaneously or in a sequence. As another variation, one or more of these steps (590, 592, 594, 596) may be omitted. Still other kinds of automated responses that may be provided in response to detection of the targeted anatomical passageway will be apparent to those of ordinary skill in the art in view of the teachings herein.

At step (590), IGS navigation system (100) uploads the particular CT scan view layout that corresponds to the particular anatomical passageway to be treated onto display screen (114). This may include one or more views showing particular cross-sectional planes and viewing angles of the targeted anatomical passageway and/or one or more pathways leading to the targeted anatomical passageway.

At step (592), IGS navigation system (100) uploads the particular clipping angle that corresponds to the particular anatomical passageway to be treated.

In some versions, dilation instrument (500) is coupled with an automated inflator system that is operable to communicate fluid to and from the dilation catheter in an automated fashion. Such an automated inflator system may be constructed and operable in accordance with at least some of the teachings of U.S. Pub. No. 2016/0058985, entitled “Automated Inflator for Balloon Dilator,” published Mar. 3, 2016, the disclosure of which is incorporated by reference herein. Step (594) is associated with versions where an automated inflator system is used. In particular, at step (594), IGS navigation system (100) uploads the particular fluid pressure profiles for use by the balloon of the dilation catheter that correspond to the particular anatomical passageway to be treated.

In some versions, IGS navigation system (100) is operable to provide active navigation instructions to the operator via display screen (114), based on the particular anatomical passageway that is being targeted. By way of example only, such active navigation instructions may be provided in accordance with at least some of the teachings of U.S. Pub. No. 2016/0008083, entitled “Guidewire Navigation for Sinuplasty,” published Jan. 14, 2016, the disclosure of which is incorporated by reference herein. Step (596) is associated with versions where active navigation instructions are provided. In particular, at step (594), IGS navigation system (100) uploads the particular guidance instructions overlaid on the CT scan view layout displayed on screen (114) based on the particular anatomical passageway to be treated.
E. Exemplary Deflectable Guide with Control having Angular Position Sensor

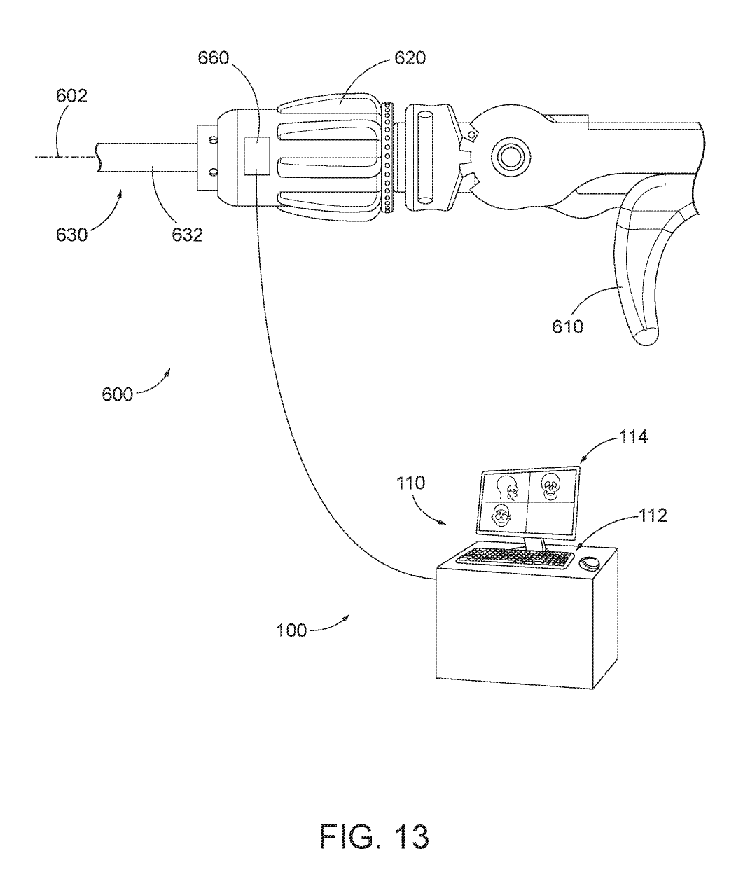

FIG. 13 shows an exemplary dilation instrument (600) that is configured to enable adjustment of an integral guide catheter for use with various different anatomical passageways. Dilation instrument (600) is configured to be operatively connected to IGS navigation system (100) via wire or wirelessly. Except as otherwise described below, dilation instrument (600) may be configured and operable just like dilation instrument (500) described above. However, dilation instrument (600) is different than dilation instrument (500) in that dilation instrument (600) includes an angular position sensor (660) rather than a linear position sensor (550) like dilation instrument (500).

Deflection control knob (620) is positioned at a distal end of handle assembly (610) and a proximal end of shaft assembly (630). Deflection control knob (620) is configured to deflect and/or laterally bend a flexible portion of shaft assembly (630) relative to handle assembly (610) in response to rotation of control knob (620) relative to handle assembly (610), just like deflection control knob (520) described above. In other words, deflection control knob (620) is rotatable about a longitudinal axis (602) defined by the longitudinal length of shaft assembly (630).

As further seen in FIG. 13, dilation instrument (600) includes an angular position sensor (660) that is operable to track rotation of control knob (620) relative to handle assembly (610). The angular position of control knob (620) will be indicative of the longitudinal position of the push-pull wire (not shown) in shaft assembly (630), which will be indicative of the bend angle of the flex section of shaft assembly (630), which will in turn be indicative of the anatomical passageway that is being targeted by dilation instrument (500). A processor in dilation instrument (600) and/or in IGS navigation system (100) (e.g., processor (110)) may process signals from angular position sensor (660) to determine the bend angle of the flex section of shaft assembly (630), and thereby determine the anatomical passageway that is being targeted by dilation instrument (600). Processor (110) is operable to upload then preset settings that correspond to treating that particular anatomical passageway.

While angular position sensor (660) is shown as being positioned at a particular location in FIG. 13, angular position sensor (660) may instead be fixedly secured anywhere that angular position sensor (660) is operable to track angular movement of control knob (620). Various suitable forms that angular position sensor (660) may take will be apparent to those of ordinary skill in the art in view of the teachings herein.
F. Exemplary Operation of Dilation Instrument with Deflectable Guide with Control having Angular Position Sensor

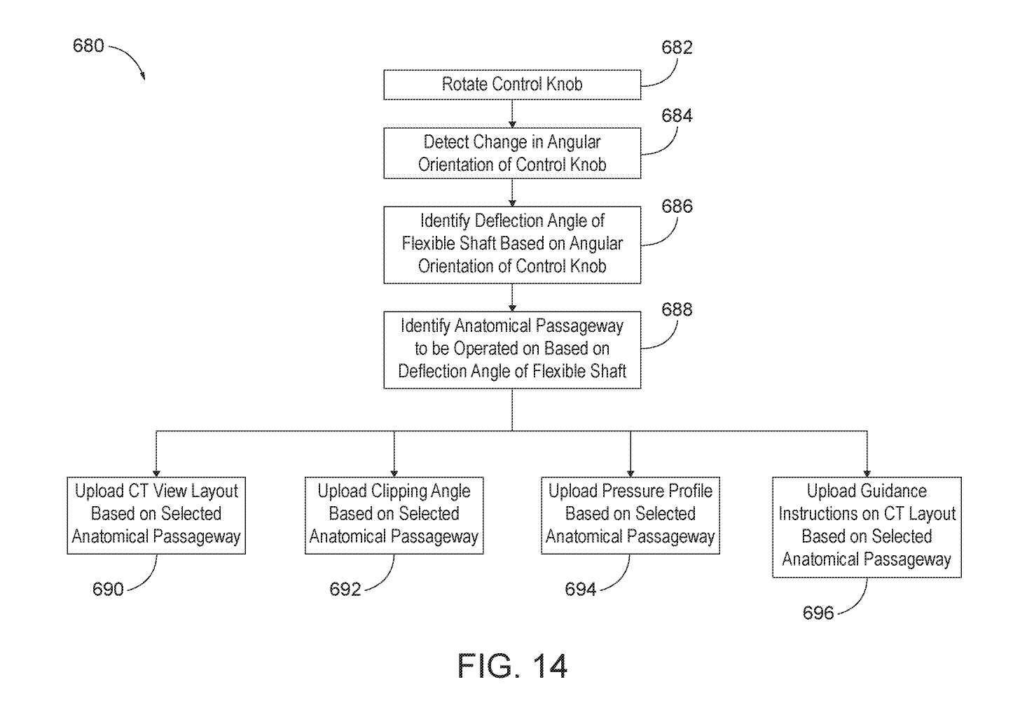

FIG. 14 shows a flow diagram illustrating steps of an exemplary method (680) that may be executed using dilation instrument (600) and IGS navigation system (100). At step (682), an operator of dilation instrument (600) determines the extent to which it is desired to deflect the flex section of shaft assembly (630). The operator rotates control knob (620) to provide deflection at the flex section of shaft assembly (630) relative to handle assembly (610), to thereby achieve a desired bend angle, as described in greater detail above.

As the operator rotates control knob (620) to achieve a desired bend angle, at step (684), angular position sensor (660) detects the change in angular position of control knob (620) relative to the fixed position of sensor (660) relative to handle assembly (610). With sensor (660) having detected the extent of angular movement of control knob (620) relative to handle assembly (610), a processor in dilation instrument (600) and/or in IGS navigation system (100) (e.g., processor (110)) determines the deflection angle formed at the flex section of shaft assembly (630) as seen at step (686). In particular, the further control knob (620) is rotated relative to handle assembly (610), the greater change in angular position that sensor (660) will detect. Accordingly, angular position sensor (660) will recognize that the extent of angular movement of control knob (620) is directly proportional to a greater bend formed at the flex section of shaft assembly (630). In this instance, as seen at step (688), a processor in dilation instrument (600) and/or in IGS navigation system (100) (e.g., processor (110)) identifies the particular type of anatomical passageway that dilation instrument (600) is being used to treat, based on the deflection angle that was identified based on data from sensor (660).

Upon receiving the data indicating which particular anatomical passageway is being targeted, processor (110) processes the information and communicates with one or more memories to upload the respective preset settings that are associated with the anatomical passageway that is to be treated. Processor (110) uses software stored in a memory to upload, calibrate, and/or operate IGS navigation system (100) with the selected preset settings. Steps (690, 692, 694, 696) show various optional responses that processor (110) may provide in response to determining which particular anatomical passageway is being targeted. These steps (690, 692, 694, 696) may be performed simultaneously or in a sequence. As another variation, one or more of these steps (690, 692, 694, 696) may be omitted. Still other kinds of automated responses that may be provided in response to detection of the targeted anatomical passageway will be apparent to those of ordinary skill in the art in view of the teachings herein.

At step (690), IGS navigation system (100) uploads the particular CT scan view layout that corresponds to the particular anatomical passageway to be treated onto display screen (114). This may include one or more views showing particular cross-sectional planes and viewing angles of the targeted anatomical passageway and/or one or more pathways leading to the targeted anatomical passageway.

At step (692), IGS navigation system (100) uploads the particular clipping angle that corresponds to the particular anatomical passageway to be treated.

In some versions, dilation instrument (600) is coupled with an automated inflator system that is operable to communicate fluid to and from the dilation catheter in an automated fashion. Such an automated inflator system may be constructed and operable in accordance with at least some of the teachings of U.S. Pub. No. 2016/0058985, entitled “Automated Inflator for Balloon Dilator,” published Mar. 3, 2016, the disclosure of which is incorporated by reference herein. Step (694) is associated with versions where an automated inflator system is used. In particular, at step (694), IGS navigation system (100) uploads the particular fluid pressure profiles for use by the balloon of the dilation catheter that correspond to the particular anatomical passageway to be treated.

In some versions, IGS navigation system (100) is operable to provide active navigation instructions to the operator via display screen (114), based on the particular anatomical passageway that is being targeted. By way of example only, such active navigation instructions may be provided in accordance with at least some of the teachings of U.S. Pub. No. 2016/0008083, entitled “Guidewire Navigation for Sinuplasty,” published Jan. 14, 2016, the disclosure of which is incorporated by reference herein. Step (696) is associated with versions where active navigation instructions are provided. In particular, at step (694), IGS navigation system (100) uploads the particular guidance instructions overlaid on the CT scan view layout displayed on screen (114) based on the particular anatomical passageway to be treated.

While dilation instruments (500, 600) are described in separate examples, some variations may include both linear position sensor (560) and angular position sensor (660), with data from both sensors (560, 660) being used to determine a bend angle and thereby determine the location of the targeted anatomical passageway. Still other suitable variations and combinations of teachings will be apparent to those of ordinary skill in the art in view of the teachings herein.
IV. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

Example 1

An apparatus, comprising: (a) body assembly, wherein the body assembly includes a first electrical interface; (b) a dilation catheter, wherein the dilation catheter includes an expandable dilator, wherein the dilation catheter is configured to translate relative to the body assembly; and (c) a first guide catheter, wherein the first guide catheter is configured to slidably receive the dilation catheter, wherein the first guide catheter comprises: (i) a first rigid shaft having a first proximal end and a first distal end, wherein the first rigid shaft has a first rigid bend formed near the first distal end, wherein the first rigid bend defines a first fixed bend angle, and (ii) a first guide catheter electrical contact located near the first proximal end, wherein the first guide catheter is configured to be removably coupled with the body assembly such that the first guide catheter electrical contact engages the first electrical interface of the body assembly; and (d) a guide catheter identification module, wherein the guide catheter identification module is operable to identify the first guide catheter based on engagement between the first guide catheter electrical contact and the first electrical interface of the body assembly.

Example 2

The apparatus of Example 1, wherein the body assembly includes a guide port having a bore, wherein the bore is configured to receive the first proximal end of the first guide catheter such that the first guide catheter projects distally from the guide port when received within the bore.

Example 3

The apparatus of Example 2, wherein the first electrical interface of the body assembly is positioned in the bore.

Example 4

The apparatus of Example 3, wherein the first proximal end of the first guide catheter includes a hub, wherein the hub is configured to be received within the bore of the guide port, wherein the first guide catheter electrical contact is fixedly secured to the hub.

Example 5

The apparatus of any one or more of Examples 1 through 4, further comprising a computing system configured to execute a series of preset settings in response to data from the guide catheter identification module identifying the first guide catheter.

Example 6

The apparatus of Example 5, wherein the series of preset settings includes at least a visual representation of an anatomical passageway associated with the first fixed bend angle.

Example 7

The apparatus of any one or more of Examples 5 through 6, wherein the series of preset settings includes at least a fluid pressure profile for use in inflating the dilator.

Example 8

The apparatus of any one or more of Examples 5 through 7, wherein the series of preset settings includes at least navigational instructions for moving the first guide catheter into position in relation to an anatomical passageway associated with the first fixed bend angle.

Example 9

The apparatus of any one or more of Examples 1 through 8, wherein the first electrical interface comprises a first pair of electrical contacts.

Example 10

The apparatus of Example 9, wherein the first guide catheter electrical contact is configured to complete a first circuit between the first pair of electrical contacts, wherein the guide catheter identification module is operable to identify the first guide catheter based on completion of the first circuit.

Example 11

The apparatus of any one or more of Examples 1 through 10, wherein the body assembly includes a second electrical interface, the apparatus further comprising a second guide catheter, wherein the second guide catheter is configured to slidably receive the dilation catheter, wherein the second guide catheter comprises: (i) a second rigid shaft having a second proximal end and a second distal end, wherein the second rigid shaft has a second rigid bend formed near the second distal end, wherein the second rigid bend defines a second fixed bend angle, and (ii) a second guide catheter electrical contact located near the second proximal end, wherein the second guide catheter is configured to be removably coupled with the body assembly such that the second guide catheter electrical contact engages the second electrical interface of the body assembly; wherein the guide catheter identification module is further operable to identify the second guide catheter based on engagement between the second guide catheter electrical contact and the second electrical interface of the body assembly.

Example 12

The apparatus of Example 11, wherein the second electrical interface is longitudinally spaced apart from the first electrical interface.

Example 13

The apparatus of any one or more of Examples 11 through 12, wherein the second electrical interface is positioned to only engage the second guide catheter electrical contact such that the second electrical interface is not capable of engaging the first guide catheter electrical contact.

Example 14

The apparatus of Example 13, wherein the first electrical interface is positioned to only engage the first guide catheter electrical contact such that the first electrical interface is not capable of engaging the second guide catheter electrical contact.

Example 15

The apparatus of any one or more of Examples 11 through 14, wherein the first fixed bend angle is configured to promote access to a first drainage passageway associated with a first paranasal sinus, wherein the second fixed bend angle is configured to promote access to a second drainage passageway associated with a second paranasal sinus.

Example 16

An apparatus comprising: (a) a body; (b) a shaft assembly extending distally from the body along a longitudinal axis, wherein a distal portion of the shaft assembly is laterally deflectable from the longitudinal axis such that the shaft assembly is configured to form a bend along the distal portion; (c) a deflection actuation assembly coupled to the shaft assembly, wherein the deflection actuation assembly is configured to laterally deflect the distal portion from the longitudinal axis in response to movement of the deflection actuation assembly; (d) a sensor configured to monitor movement associated with the deflection actuation assembly to thereby measure a bend angle defined by the bend formed along the distal portion; and (e) a bend angle detection module, wherein the bend angle detection module is operable to identify the formed bend angle.

Example 17

The apparatus of Example 16, wherein the deflection actuation assembly comprises a linearly translating member that is configured to translate, wherein the sensor is operable to monitor longitudinal displacement of the linearly translating member.

Example 18

The apparatus of any one or more of Examples 16 through 17, wherein the deflection actuation assembly comprises a rotary member that is configured to rotate, wherein the sensor is operable to monitor angular displacement of the rotary member.

Example 19

A method comprising assembling a guide catheter onto a dilation instrument body such that an electrical contact of the dilation instrument body encounters a corresponding electrical contact of the guide catheter, wherein the guide catheter defines a bend angle associated with a particular anatomical passageway of a patient; wherein a processor automatically identifies the anatomical passageway associated with bend angle of the guide catheter, based on engagement between the electrical contact of the dilation instrument body and the corresponding electrical contact of the guide catheter; wherein the processor automatically uploads one or more programmed settings associated with the identified anatomical passageway.

Example 20

The method of Example 20, further comprising: (a) inserting the guide catheter into a patient; (b) guiding a dilator into the particular anatomical passageway of the patient via the guide catheter; and (c) actuating the dilator to dilate the particular anatomical passageway of the patient.

Example 21

The method of Example 20, wherein the act of inserting the guide catheter into a patient comprises inserting the guide catheter into a nasal cavity of the patient.

Example 22

The method of Example 21, wherein the act of guiding a dilator into the particular anatomical passageway of the patient via the guide catheter comprises guiding the dilator into a drainage passageway associated with a paranasal sinus, wherein the bend angle of the guide catheter is configured to facilitate access to the drainage passageway associated with the paranasal sinus.

Example 23

The method of Example 21, wherein the act of guiding a dilator into the particular anatomical passageway of the patient via the guide catheter comprises guiding the dilator into a Eustachian tube, wherein the bend angle of the guide catheter is configured to facilitate access to the Eustachian tube.

Example 24

The method of any one or more of Examples 19 through 23, wherein the particular anatomical passageway of the patient is in the head of the patient, wherein the processor automatically uploads a cross-sectional view of the head of the patient at a cross-sectional plane associated with the particular anatomical passageway in the head of the patient.
V. Miscellaneous

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices disclosed herein can be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, versions of the device may be disassembled, and any number of the particular pieces or parts of the device may be selectively replaced or removed in any combination. Upon cleaning and/or replacement of particular parts, versions of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a surgical facility. A device may also be sterilized using any other technique known in the art, including but not limited to beta or gamma radiation, ethylene oxide, or steam.

Having shown and described various versions of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus, comprising:
(a) body assembly, wherein the body assembly includes a first electrical interface;
(b) a dilation catheter, wherein the dilation catheter includes an expandable dilator, wherein the dilation catheter is configured to translate relative to the body assembly; and
(c) a first guide catheter, wherein the first guide catheter is configured to slidably receive the dilation catheter, wherein the first guide catheter comprises:
(i) a first rigid shaft having a first proximal end and a first distal end, wherein the first rigid shaft has a first rigid bend formed near the first distal end, wherein the first rigid bend defines a first fixed bend angle, and
(ii) a first guide catheter electrical contact located near the first proximal end, wherein the first guide catheter is configured to be removably coupled with the body assembly such that the first guide catheter electrical contact engages the first electrical interface of the body assembly; and
(d) a guide catheter identification module, wherein the guide catheter identification module is operable to identify the first guide catheter based on engagement between the first guide catheter electrical contact and the first electrical interface of the body assembly.

2. The apparatus of claim 1, wherein the body assembly includes a guide port having a bore, wherein the bore is configured to receive the first proximal end of the first guide catheter such that the first guide catheter projects distally from the guide port when received within the bore.

3. The apparatus of claim 2, wherein the first electrical interface of the body assembly is positioned in the bore.

4. The apparatus of claim 3, wherein the first proximal end of the first guide catheter includes a hub, wherein the hub is configured to be received within the bore of the guide port, wherein the first guide catheter electrical contact is fixedly secured to the hub.

5. The apparatus of claim 1, further comprising a computing system configured to execute a series of preset settings in response to data from the guide catheter identification module identifying the first guide catheter.

6. The apparatus of claim 5, wherein the series of preset settings includes at least a visual representation of an anatomical passageway associated with the first fixed bend angle.

7. The apparatus of claim 5, wherein the series of preset settings includes at least a fluid pressure profile for use in inflating the dilator.

8. The apparatus of claim 5, wherein the series of preset settings includes at least navigational instructions for moving the first guide catheter into position in relation to an anatomical passageway associated with the first fixed bend angle.

9. The apparatus of claim 1, wherein the first electrical interface comprises a first pair of electrical contacts.

10. The apparatus of claim 9, wherein the first guide catheter electrical contact is configured to complete a first circuit between the first pair of electrical contacts, wherein the guide catheter identification module is operable to identify the first guide catheter based on completion of the first circuit.

11. The apparatus of claim 1, wherein the body assembly includes a second electrical interface, the apparatus further comprising a second guide catheter, wherein the second guide catheter is configured to slidably receive the dilation catheter, wherein the second guide catheter comprises:
(i) a second rigid shaft having a second proximal end and a second distal end, wherein the second rigid shaft has a second rigid bend formed near the second distal end, wherein the second rigid bend defines a second fixed bend angle, and
(ii) a second guide catheter electrical contact located near the second proximal end, wherein the second guide catheter is configured to be removably coupled with the body assembly such that the second guide catheter electrical contact engages the second electrical interface of the body assembly;
wherein the guide catheter identification module is further operable to identify the second guide catheter based on engagement between the second guide catheter electrical contact and the second electrical interface of the body assembly.

12. The apparatus of claim 11, wherein the second electrical interface is longitudinally spaced apart from the first electrical interface.

13. The apparatus of claim 11, wherein the second electrical interface is positioned to only engage the second guide catheter electrical contact such that the second electrical interface is not capable of engaging the first guide catheter electrical contact.

14. The apparatus of claim 13, wherein the first electrical interface is positioned to only engage the first guide catheter electrical contact such that the first electrical interface is not capable of engaging the second guide catheter electrical contact.

15. The apparatus of claim 11, wherein the first fixed bend angle is configured to promote access to a first drainage passageway associated with a first paranasal sinus, wherein the second fixed bend angle is configured to promote access to a second drainage passageway associated with a second paranasal sinus.

16. An apparatus comprising:
(a) a body;
(b) a shaft assembly extending distally from the body along a longitudinal axis, wherein a distal portion of the shaft assembly is laterally deflectable from the longitudinal axis such that the shaft assembly is configured to form a bend along the distal portion;
(c) a deflection actuation assembly coupled to the shaft assembly, wherein the deflection actuation assembly is configured to laterally deflect the distal portion from the longitudinal axis in response to movement of the deflection actuation assembly;
(d) a sensor configured to monitor movement associated with the deflection actuation assembly to thereby measure a bend angle defined by the bend formed along the distal portion; and
(e) a bend angle detection module, wherein the bend angle detection module is operable to identify the formed bend angle.

17. The apparatus of claim 16, wherein the deflection actuation assembly comprises a linearly translating member that is configured to translate, wherein the sensor is operable to monitor longitudinal displacement of the linearly translating member.

18. The apparatus of claim 16, wherein the deflection actuation assembly comprises a rotary member that is configured to rotate, wherein the sensor is operable to monitor angular displacement of the rotary member.

19. A method comprising assembling a guide catheter onto a dilation instrument body such that an electrical contact of the dilation instrument body encounters a corresponding electrical contact of the guide catheter, wherein the guide catheter defines a bend angle associated with a particular anatomical passageway of a patient;
wherein a processor automatically identifies the anatomical passageway associated with bend angle of the guide catheter, based on engagement between the electrical contact of the dilation instrument body and the corresponding electrical contact of the guide catheter;
wherein the processor automatically uploads one or more programmed settings associated with the identified anatomical passageway.

20. The method of claim 19, further comprising:
(a) inserting the guide catheter into a patient;
(b) guiding a dilator into the particular anatomical passageway of the patient via the guide catheter; and
(c) actuating the dilator to dilate the particular anatomical passageway of the patient.
